(19) United States
(12) Patent Application Publication
(10) Pub. No.: US 2026/0047768 A1
(43) Pub. Date: Feb. 19, 2026

(54) Integrated Electrocardiographic Mobile Complex

(71) Applicants: IVAN MELNIKAU, Gomel (BY); ALEXANDER DANILOV, Aventura, FL (US)

(72) Inventors: IVAN MELNIKAU, Gomel (BY); ALEXANDER DANILOV, Aventura, FL (US)

(21) Appl. No.: 19/283,699

(22) Filed: Jul. 29, 2025

Related U.S. Application Data
(60) Provisional application No. 63/683,035, filed on Aug. 14, 2024.

(51) Int. Cl. A61B 5/0205; A61B 5/00; A61B 5/024; A61B 5/0531; A61B 5/11; A61B 5/1455; A61B 5/332; G01J 5/00; G01J 5/34

(52) U.S. Cl. CPC A61B 5/02055; A61B 5/0006; A61B 5/002; A61B 5/0531; A61B 5/1118; A61B 5/14552; A61B 5/332; A61B 5/4561; A61B 5/7207; A61B 5/7225; A61B 5/742; A61B 5/7455; A61B 5/746; G01J 5/0025; G01J 5/34; A61B 5/02427; A61B 2560/0209; A61B 2560/0214; A61B 2560/0431; A61B 2560/0456; A61B 2560/0475; A61B 2562/0219; A61B 2562/0271

(57) ABSTRACT

An integrated electrocardiographic mobile complex includes a housing with embedded electrodes and a microcontroller comprising a cardiomodule, pulse oximeter module, impedance measurement module, digital accelerometer, temperature measurement module, light indicator module, and tactile indicator module. An analog-to-digital converter is configured to receive analog signals from the electrodes and convert them into digital signals. The system enables synchronized acquisition of electrocardiographic, photoplethysmographic, pulse oximetry, and bioimpedance data. A memory unit stores the acquired data, and a wireless transmit/receive unit comprising a Bluetooth module and an RF module enables wireless communication with an external device at distances of at least one kilometer. The pulse oximeter module supports low-noise signal processing and can be placed in a standby mode via software while remaining electrically connected. The invention enables real-time, multi-parameter physiological diagnostics in a portable and power-efficient form factor.

### CROSS-REFERENCE TO RELATED APPLICATION

This application claims the benefit of U.S. Provisional Application No. 63/683,035 filed on Aug. 14, 2024, which is incorporated herein by reference in its entirety.

### FIELD OF INVENTION

The present invention pertains to the field of biomedical monitoring devices, systems and methods specifically to an integrated electrocardiographic mobile complex designed for real-time health monitoring. The device and method are capable of acquiring, processing, and wirelessly transmitting applicable data, including electrocardiographic (ECG) signals, pulse oximetry, photoplethysmography, and bioimpedance measurements. This invention facilitates comprehensive and continuous monitoring of a patient's cardiovascular and respiratory functions through a compact and mobile platform.

### BACKGROUND OF THE INVENTION

Conventional systems for monitoring cardiac and physiological parameters often involve multiple discrete components, wired connections, and limited mobility. These systems typically lack real-time synchronization across various physiological metrics and often cannot transmit data wirelessly over long distances, reducing their utility in remote or ambulatory settings. There is a need for a compact, mobile, and functionally integrated system that enables multi-parameter monitoring with high data fidelity and long-range communication capability.

According to Russian Patent No. 2268641 differential vectorcardiograph is currently in use, wherein the electrical activity of the heart is measured by representing the study results as a large variety of real-time vectorcardiograms.

The device contains an HF generator and a common electrode, electrodes fixed on the human body, and a calculator of the electrical activity of the heart connected in series. For this purpose, the differential vectorcardiograph incorporates an electrocardiogram and rheoelectrogram recording unit containing amplification and filtering channels, whose inputs are connected to the corresponding electrodes located in a plane array on the surface of the chest. The outputs of the above unit are the outputs of the electrocardiogram and rheoelectrogram recording unit and are connected in series with a multiplexer, whose input is connected to the output of the electrocardiogram and rheoelectrogram recording unit and analogue-to-digital converter (ADC). The calculator of the electrical activity of the heart is designed as a microprocessor unit with a common bus to which the multiplexer control input, ADC output, keyboard output, mouse output and indicator module input are connected.

A drawback of the cardiograph that is currently in use is that it can be used only in stationary conditions, which makes it difficult to use it for real-time monitoring of the human health status. Another drawback is functionally limited data collecting, in particular on the blood oxygen level in the process of testing or, for example, evaluation of the cardiovascular system status on exertion.

Russian Patent No. 2738862 discloses a hardware-software complex for diagnosing the state of the cardiovascular system using a personal computer (PC) with appropriate software. When diagnosing, an electrocardiogram (ECG), blood pressure (BP) and ankle-brachial index (ABI) are recorded using the parameter recording unit of the hardware-software complex. In addition, a sphygmogram is recorded to perform volumetric sphygmography and apex cardiography (ApexCG). A rheogram is also recorded using the same parameter recording unit. The parameter recording unit comprises an ECG recording module, a rheogram recording module, and a sphygmogram recording module providing volumetric sphygmography. The hardware-software complex also includes an apexcardiogram recording module, two modules of non-invasive blood pressure measurement with the possibility of cuff pumping, a microcontroller and a communication module. Then, the data from the recording unit, as well as the data of medical history and physical examination, are transferred to the PC. The received information, together with the cloud data, is processed, and the primary diagnosis and treatment recommendations are given to the cardiologist.

A drawback of the analogue that is currently in use is that the complex cannot be used by the individual user at home, which makes it difficult to use it for real-time monitoring of the human health status. Another drawback is lacking functions of monitoring the blood oxygen level in the process of testing and evaluating the cardiovascular system status on exertion.

Korean Patent Publication No. 1020190091880 Teaches a Mobile glove-shaped heart rate monitor. The device is designed to be worn on the user's hand for measuring heart rate. It comprises the following elements: an input unit for a training mode appropriate for the user's health status; a speaker for audio signals in which the number of beats per time unit changes according to the training mode; a heart rate sensor that is attached to the user's finger and measures the user's heart rate for generating a sensor signal; a control unit for extracting the user's heart rate from the sensor signal received from the heart rate sensor; and a visual display unit. The control unit sets the user's normal heart rate range according to the training mode, compares the determined heart rate and the obtained heart rate to control the speaker to output a warning beep and control the display unit to output a visual warning if the determined heart rate is beyond the normal heart rate range. The heart rate sensor is positioned on the finger of the glove. As claimed in the patent claim, the hazard level is displayed by means of an LED to recognise the hazard, and when a real hazardous condition is suspected, a warning beep is on to allow the user to become aware of the real hazardous condition. Hence, low-risk patients in cardiac rehabilitation can exercise safely.

US Patent Publication 2006122525 Discloses a System for Non-contact recording of electrocardiographic and magnetocardiographic parameters of human physical activity designed as a portable medical device. The device records body position, respiratory rate, temperature, blood pressure, vasomotor activity, blood flow, neural activity and other physiological data about the user's health status. During its operation, the system retrieves and displays the most significant parameters from time series of the above data. The system achieves the desired sensitivity in signal-to-noise ratio for the miniaturised device by collecting data from at least one control point of the cardiac complex over a period of at least one, and preferably several seconds, and displays the underlying typical patterns of the data. By means of miniaturisation (the device is pocket-sized), the system can be made, for example, in the form of a pen or other miniature object that can be carried in a user's pocket.

A drawback of the above-noted miniature analogues of the devices is insufficient functional application due to the impossibility of controlling the blood oxygen level, which decreases the consumer value of such mobile devices.

Closes prior art known to the inventors relative to the present invention is Russian Patent No. 2759404 which discloses a hardware-software electrocardiographic measuring complex. The hardware-software ECG complex comprises a housing, fixed on the user, wherein the following are installed: a microcontroller that controls an electronic device; an analogue-to-digital converter converting analogue signals coming from the electrodes to digital ones; a data storage unit made with the possibility to store ECG data; a wireless transmit/receive module providing data transfer from the memory unit to an external computing device; the first electrode combined with a capacitive button activating the ECG recording function of the device; the second and third electrodes positioned on the rear side of the housing and providing ECG recording together with the first electrode; external electrode connectors for ECG recording. The bandwidth of frequency-response characteristics for all analogue interface channels is 0 to 4,000 Hz, acceptable amplitude deviation is 0 to 2,000 Hz ±10%, signal sampling rate is 8,000 Hz. The microcontroller and data storage unit are designed to record, read and transmit ECG data in parallel streams with priority to the recording process, with recording to non-volatile memory in single intervals in a size multiple of the page size of the non-volatile memory. The microcontroller is designed to implement direct diagnostic algorithms based on rules and decision trees. The microcontroller is designed to be connected to diagnostic units implementing machine learning algorithms. The hardware-software ECG complex is made with the possibility to be connected to the external electrode connector with a 7-pin cable allowing to record a standard 12-lead ECG. The hardware-software ECG complex operates as follows. All recorded ECGs are subjected to automatic analysis using key point detection (KPD), which are segmented and automatically described in the same pre-medical report format by the developed software according to conventional ECG analysis criteria. The algorithm of the KPD method application includes the following steps: ECG signal pre-processing, filtering (noise suppression), isoline extraction, detection of the KPD signal onset, peak and end of the QRS complex, P-and T-waves, determination of their morphology, and automatic generation of a conclusion.

A drawback of Russian Patent No. 2759404 is the narrow functionality of the hardware-software complex due to recording and processing of ECG signals only. Another drawback is low-speed conversion of analogue signals coming from the electrodes into digital ones, as the operating frequency of the analogue-to-digital converter does not exceed 4 kHz. It is also a drawback that it is not possible to obtain data on the user's heart rate in dynamic mode, in particular, during athletic training.

An essential limitation of Russian Patent No. 2759404n is the restricted functionality of the hardware-software system, which is confined solely to the recording and processing of ECG signals. Additionally, the system suffers from a low-speed conversion of analog signals from the electrodes to digital format, as the analog-to-digital converter operates at a frequency not exceeding 4 kHz. Another significant drawback is the inability to capture heart rate data in dynamic conditions, such as during physical activity or athletic training.

Thus, it has been long felt and unsolved need for a compact, mobile, and functionally integrated system that enables multi-parameter monitoring with high data fidelity and long-range communication capability.

### SUMMARY OF THE INVENTION

One of the objectives of the invention is to eliminate the mentioned has been long felt drawbacks and to improve the consumer properties of the device and method.

An essential objective of the invention is to provide comprehensive monitoring of the human body state in dynamic mode with obtaining the results of ECG examination, determination of the real-time blood oxygen level to prevent complications or critical conditions of the cardiovascular system.

An essential objective of the invention is achieved due to the fact that the portable hardware-software ECG complex is provided consisting of a housing with electrodes installed therein and a microcontroller including an analogue-to-digital converter converting analogue signals coming from the electrodes for implementing diagnostics, a memory unit for storing data, a wireless transmit/receive unit for transferring data from the memory unit of the microcontroller to an external device and power supply unit, the microcontroller is made with the possibility to implement diagnostics with obtaining real-time synchronised complex electrocardiographic data, pulse oximetry, photoplethysmography and bioimpedancemetry, wherein the microcontroller comprises a functionally connected cardiomodule, a pulse oximeter module, an impedance measurement module, a digital accelerometer, a temperature measurement module, a light indicator module and a tactile indication module, wherein the wireless transmit/receive unit is made as a combined one containing a Bluetooth module and an RF module as transceivers; one of the electrodes on the front surface of the housing is functionally connected with the cardiomodule for recording electrical signals of the heart, and the other electrode contains a sensor connected with the pulse oximeter module, made with the possibility of parallel recording of optical signals to measure the SpO2 level (blood oxygen level); the analogue-to-digital converter is made with the possibility of receiving an analogue signal from the cardiomodule and converting it into a digital signal, as well as receiving digital data from the pulse oximeter module, impedance measurement module and temperature sensor with subsequent transfer of the collected data into a data package to the transmit/receive unit, the pulse oximeter module is equipped with optimised optics that provide low-noise analogue signal processing for detecting pulse oximetry signals, and is made with the possibility of software shutdown in standby mode while maintaining constant mode of connection of the power supply unit; the RF module of the wireless transmit/receive unit is made with the possibility of transmitting/receiving electrical signals at a distance of at least one kilometre away from an external receiving device with generating up to one hundred time-and-frequency communication channels.

According to a fundamental aspect, the present invention provides a portable hardware-software electrocardiographic (ECG) system and, more specifically, to a multifunctional diagnostic device capable of real-time monitoring and recording of complex physiological parameters including electrocardiographic signals, blood oxygen saturation (SpO₂), photoplethysmographic data, bioimpedance measurements, and temperature.

The system comprises a compact housing in which at least two electrodes are integrated. The housing accommodates a microcontroller, a memory unit, a wireless data transmission unit, and a power supply unit. The microcontroller is equipped with an analog-to-digital converter (ADC) and is configured to receive, process, and digitize analog signals obtained from the electrodes and other sensors.

Further, the microcontroller is functionally configured to implement diagnostic operations and is capable of acquiring synchronized, real-time physiological data. The microcontroller includes a plurality of functional modules, specifically:
A cardiomodule for detecting and processing electrocardiographic signals;
A pulse oximeter module configured to measure blood oxygen saturation levels (SpO₂) and pulse rate;
An impedance measurement module for bioimpedance analysis;
A digital accelerometer for motion and orientation tracking;
A temperature measurement module;
A light indication module for visual status alerts;
A tactile feedback module for haptic notifications.

Still further, the electrodes are positioned such that at least one electrode on the front surface of the housing is electrically connected to the cardiomodule for acquiring cardiac electrical signals. Another electrode integrates a sensor that interfaces with the pulse oximeter module, allowing simultaneous optical signal detection used for pulse oximetry and photoplethysmography.

The analog-to-digital converter of the invention is designed to receive analog signals from the cardiomodule and convert them into corresponding digital signals. It is also configured to accept digital input data from the pulse oximeter module, the impedance measurement module, and the temperature sensor. The ADC or associated controller aggregates this multisource physiological data into structured data packets for wireless transmission.

The wireless communication subsystem of the invention comprises a dual-mode transceiver including a Bluetooth module and a radio frequency (RF) module. This configuration supports short-range (Bluetooth) and long-range (RF) data transmission. The RF module is capable of transmitting and receiving signals over a distance of at least one kilometer from a compatible external receiving device. Moreover, the RF module supports up to 100 distinct time-and-frequency division communication channels, allowing for robust, interference-resistant data transfer in diverse environments.

The pulse oximeter module of the invention features optoelectronic components and signal processing circuitry optimized to reduce analog signal noise, thereby enhancing measurement accuracy. The module is further designed to support a low-power standby mode, in which software-controlled shutdown is enabled while maintaining continuous connection to the power supply unit, thereby improving energy efficiency without compromising readiness.

The power supply unit of the invention is configured to provide consistent and regulated power to all internal modules and may include a rechargeable battery with integrated power management circuitry.

The memory unit is made SPI (Serial Peripheral Interface) non-volatile interface with the possibility of storing data in the absence of communication between the portable hardware-software ECG complex and external devices: a receiving docking station or mobile phone.

The cardiomodule is made as an analogue interface with the possibility of measuring the patient's biopotentials, subsequent signal amplification and filtering under conditions of high noise, such as artifacts of the patient's muscle movements and external electromagnetic interference from a single lead of the cardiogram.

The impedance measurement module is configured with the possibility of providing measurements of the patient's body complex impedance values via SPI bus and transferring the data to the microcontroller.

The temperature measurement module is formed based on a digital integrated circuit with the possibility to measure contact or non-contact temperature on the patient's fingers.

The pulse oximeter module is configured based on a digital integrated circuit and contains an optical interface with the possibility of displaying information on pulse wave and blood oxygen level.

The tactile indication module comprises an oscillation generator as a source of tactile information that is connected to the housing and made with the possibility of receiving information by the user during active physical activity or when other information about the device status is not available.

The light indicator module comprises RGB (RGB stands for red, green, and blue) LEDs to display information on the status of the portable hardware-software ECG complex and its operating modes.

One of the essential aspects of the present invention relates to an integrated electrocardiographic mobile complex designed for comprehensive real-time physiological monitoring. The device includes a compact housing incorporating multiple physiological sensing modules and wireless communication capabilities, enabling continuous, mobile health monitoring in both clinical and non-clinical environments.

The system features at least one electrode disposed on the housing for detecting cardiac signals, which are processed by a cardiomodule integrated into a microcontroller within the housing. The microcontroller includes an analog-to-digital converter that converts analog signals from the cardiomodule into digital form. In addition to cardiac signal processing, the system includes a pulse oximeter module that receives and processes optical signals for determining blood oxygen saturation (SpO₂) levels.

Additional measurements are supported by an impedance measurement module, a digital accelerometer, and a temperature measurement module comprising a temperature sensor. The device also incorporates a light indicator module and a tactile indication module to provide visual and haptic feedback to the user.

Collected data from all sensing modules are stored in a memory unit and managed by the microcontroller, which is configured to perform diagnostics and synchronize real-time data from multiple physiological sources including electrocardiography, pulse oximetry, photoplethysmography, and bioimpedance analysis. This data is compiled and transferred as a unified data package to a wireless transmit/receive unit.

The wireless communication system includes both a Bluetooth module for short-range data exchange and an RF module capable of long-range data transmission (at least one kilometer), supporting up to one hundred time-and-frequency communication channels. This enables seamless integration with external devices for data review, storage, or remote diagnostics.

One of the electrodes on the front surface of the housing is connected to the cardiomodule for receiving electrocardiographic signals, while another electrode integrates an optical sensor for pulse oximetry, allowing for the parallel recording of electrical and optical signals. The pulse oximeter module is further configured to process low-noise analog signals and to operate in a software-controlled standby mode while maintaining a continuous power supply connection, thereby enhancing power efficiency.

Overall, the invention provides a multifunctional, portable, and highly integrated physiological monitoring solution capable of real-time data acquisition and wireless communication, supporting a broad range of health monitoring applications.

In summary, the disclosed portable ECG system offers a comprehensive, real-time physiological monitoring solution. Its modular design enables the integration of multiple diagnostic functions into a single compact device, while the combination of Bluetooth and RF wireless communication facilitates flexible data sharing across both local and remote distances.

Further important aspect of the present invention relates to a method for performing medical diagnostics using an integrated electrocardiographic mobile complex. The system includes a mobile diagnostic unit comprising a housing that contains electrodes, a microcontroller, memory, a wireless communication unit, and a power supply. The mobile complex is configured to receive electrical signals from at least one electrode placed on the subject's skin to obtain electrocardiographic data. These analog signals are processed within a cardiomodule and converted to digital format for analysis.

In addition to cardiac data, the system collects optical signals through a sensor associated with a pulse oximeter module to determine blood oxygen saturation (SpO₂) levels. It also acquires physiological data from a bioimpedance measurement module, a digital accelerometer, and a temperature sensor.

All data are digitized, aggregated into a comprehensive physiological data package, and transmitted wirelessly to an external device. Wireless communication is achieved through integrated Bluetooth and RF modules. This mobile system allows for real-time, multi-parametric physiological monitoring in a compact and portable format.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

The invention may best be understood by reference to the following detailed description when read with the accompanying drawings, wherein:

FIG. 1 shows a general view of the device.

FIG. 2 shows a schematic block diagram of the hardware-software electro cardio graphic measuring complex.

FIG. 3 shows a display view of the receiving device along with pulse wave and heart rate diagrams.

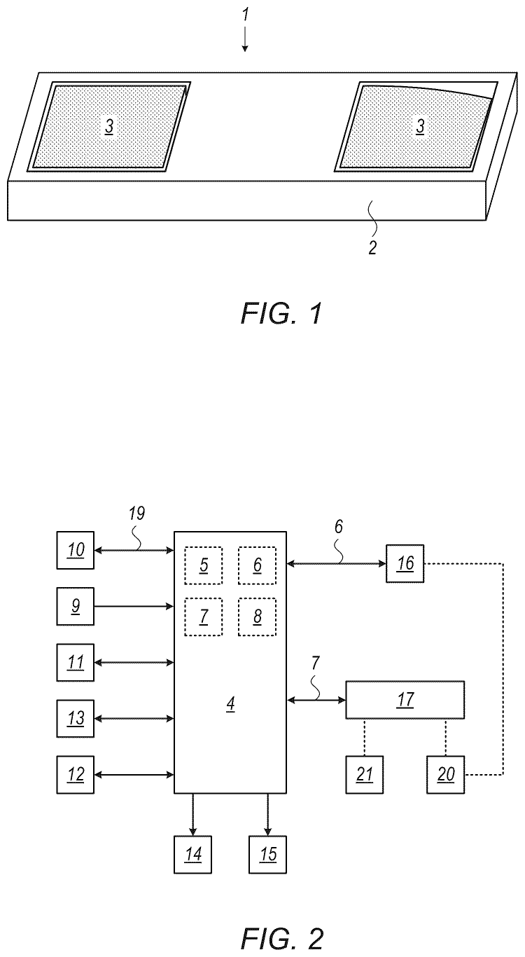

### DESCRIPTION OF EMBODIMENTS OF THE INVENTION

In the following detailed description, numerous specific details are set forth in order to provide a thorough understanding of the invention. However, it will be understood by those skilled in the art that the invention may be practiced without these specific details. In other instances, well-known methods, procedures, and components have not been described in detail so as not to obscure the invention.

The hardware-software ECG complex (1) comprises a housing (2) having electrodes (3) disposed on its front surface (18) for acquiring bioelectrical signals. Located within the housing (2) is a microcontroller (4) that includes an analog-to-digital converter (5) for converting analog physiological signals into digital data, a memory unit (6) for storing the acquired data, and a wireless transmit/receive unit (7) for communication with external devices. The wireless transmit/receive unit (7) comprises a Bluetooth module (16) and an RF module (17), both configured as transceivers. The microcontroller (4) is further connected to a power supply unit (8) for providing power to the components, as well as functionally and kinematically connected to a cardiomodule (9), a pulse oximeter module (10), an impedance measurement module (11), a digital accelerometer (12), a temperature measurement module (13), a light indicator module (14), and a tactile indication module (15). The ECG complex (1) is also configured to interface with external devices, including a mobile phone (20) and a docking station (21), for purposes such as data transmission, device control, charging, and extended functionality.

The hardware component of the portable hardware-software ECG complex (1) is configured in accordance with applicable technical regulations. As shown in the drawings, the complex (1) comprises a housing (2) in which a microcontroller board (4) is mounted. A plurality of electrodes (3) is disposed on a front surface (18) of the housing (2) to allow for contact with the user. A power supply unit (8), preferably a lithium-ion battery, is also enclosed within the housing (2) to provide electrical power to the system components. The arrangement of the microcontroller (4), electrodes (3), and power supply unit (8) within and on the housing (2) enables compact and efficient operation of the ECG complex (1) in a portable form factor.

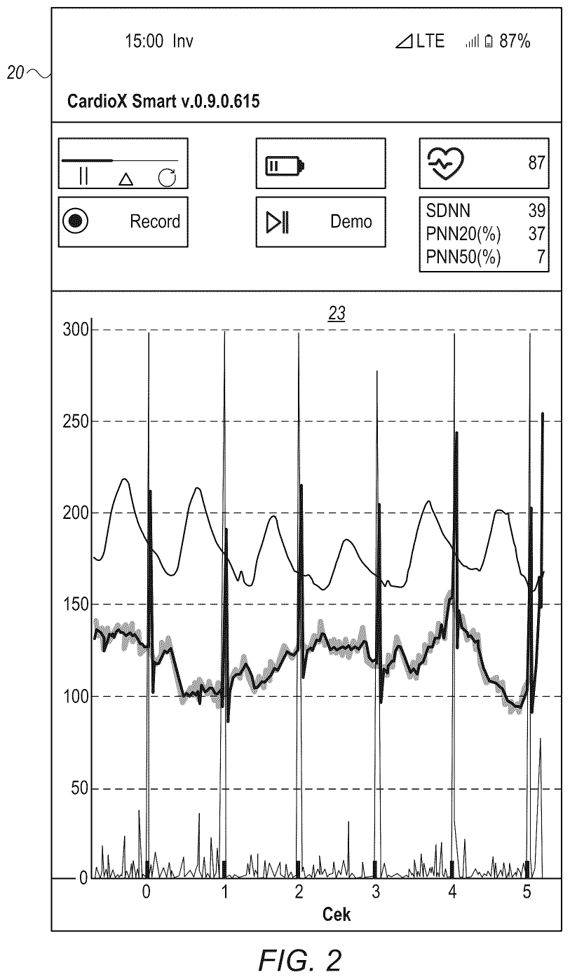

As illustrated in FIG. 2, the microcontroller (4) may be implemented as a 32-bit processor with a core clock speed of at least 72 MHz, or alternatively as a 64-bit processor offering comparable functionality. A memory block (6), preferably non-volatile, is connected to the microcontroller (4) and may be realized using an integrated circuit such as the W25Q128JVEIQ or an equivalent SPI interface memory, thereby enabling data storage in the absence of communication between the portable hardware-software ECG complex (1) and external devices, such as a receiving docking station (20) and/or a mobile phone (21). The microcontroller (4) further includes a cardiomodule (9), which functions as an analog interface, and may be implemented using an integrated circuit such as the AD8232 or an equivalent. The cardiomodule (9) is configured to detect and measure the biopotentials of a patient, amplify the signal, and apply filtering to minimize noise caused by muscle artifacts and external electromagnetic interference, all from a single ECG lead.

An impedance measurement module (11), also connected to the microcontroller (4), may be based on an integrated circuit such as the AD5940 or a functional equivalent. This module (11) is capable of measuring the complex impedance of the patient's body through communication over the SPI bus and transmits the corresponding data to the microcontroller (4). A temperature measurement module (13) is further included and may be implemented using a digital temperature sensor such as the DS18B20 or an equivalent digital pyrometer. The temperature measurement module (13) enables both contact and non-contact temperature measurement from the patient's fingers.

Additionally, the microcontroller (4) is operatively connected to a pulse oximeter module (10), which may be realized using a digital integrated circuit such as the MAX30100 or a functionally similar device. The pulse oximeter module (10) includes an optical interface configured to measure and display pulse waveform and blood oxygen saturation levels. For user feedback, a tactile indication module (15) is provided and includes an oscillation generator that serves as a tactile signal source. This module (15) is mounted to the housing (2) and is designed to convey information to the user, especially during active physical activity or when visual indicators are inaccessible. A light indicator module (14) is also included and may incorporate RGB LEDs (not shown in the figures) to visually indicate the operational status and various modes of the portable hardware-software ECG complex (1), as further illustrated in FIG. 3.

Following the assembly of the hardware component of the hardware-software ECG complex (1), herein referred to as “the device,” a dedicated software application is downloaded and activated on one or more external devices, such as a mobile phone (21) and a docking station (20). Upon software installation, the hardware-software complex (1) undergoes a testing procedure to verify operational readiness and initiate diagnostic functions. During testing, diagnostic algorithms are executed to collect and analyze synchronized, real-time physiological data from the user.

Specifically, electrocardiographic data is acquired using the cardiomodule (9), which enables the recording of an electrocardiogram (ECG), as well as the determination of heart rate and the regularity of heartbeats. Simultaneously, the pulse oximeter module (10) processes low-noise analog signals to obtain pulse oximetry parameters, including blood oxygen saturation and photoplethysmography (PPG) data—such as information about the condition of the vascular bed, capillary health, and pulse wave contour. In standby mode, the pulse oximeter module (10) is automatically deactivated via software control, while continuous power connection from the power supply unit (8) is maintained, thereby enhancing the energy efficiency and autonomy of the hardware-software complex (1).

The impedance measurement module (11) collects bioimpedance data, which provides insights into various body composition parameters, including fat mass, fat-free mass, cellular mass, skeletal muscle mass, as well as total body water volume and its distribution. Concurrently, the temperature measurement module (13) measures the user's body temperature using, for example, a digital pyrometer sensor (not shown). Additionally, a digital accelerometer (12) monitors the presence or absence of hand tremors, offering further insight into the user's physiological state during diagnostic evaluation.

Electrical signals associated with cardiac activity and optical signals related to blood oxygen levels are detected using a pair of electrodes (3) disposed on the front surface (18) of the hardware-software ECG complex (1). These analog electrical and optical signals are routed to an analog-to-digital converter (5) integrated within the microcontroller (4), where they are digitized for further processing. The resulting digital signals are stored in a non-volatile memory unit (6), from which the data are subsequently transmitted to external devices—specifically, a mobile phone (21) and/or a docking station (20).

Wireless communication is facilitated by a wireless transmit/receive unit (7), which comprises a Bluetooth module (16) and a radio frequency (RF) module (17), allowing for reliable, real-time data transfer. The transmitted signals are processed by the external device, and key indicators such as heart signals and blood oxygen levels are visually presented to the user via a display (23) of the mobile phone (21), as shown in FIG. 3. In parallel, signal activity and operational status may also be indicated through the light indicator module (14) of the hardware-software complex (1), providing local visual feedback during use.

The RF module (17), which forms part of the wireless transmit/receive unit (7), is configured to receive and transmit electrical signals over a range of at least one kilometer to and from an external device, such as the docking station (20). The RF module (17) is further capable of generating up to one hundred distinct time-and-frequency communication channels, thereby enabling simultaneous data communication and monitoring for up to one hundred individual users. This functionality is particularly beneficial in scenarios involving group monitoring, such as during athletic training or competitive events.

In such use cases, an athlete secures the hardware portion of the hardware-software ECG complex (1) directly to the body. The tactile indication module (15) is used to provide haptic feedback to the user, conveying physiological status information via tactile cues. Analog signals associated with the user's physiological state are acquired by the system and converted into digital signals by the analog-to-digital converter (5) integrated within the microcontroller (4). These digitized signals are then stored in the memory unit (6), from which they may be transmitted to external devices for monitoring, analysis, or display.

The integrated electrocardiographic mobile system, or portable hardware-software ECG complex (1), is designed to provide a synergistic effect through comprehensive, real-time monitoring of the physiological status of the human body under dynamic conditions. The system enables simultaneous acquisition and analysis of multiple diagnostic parameters, including electrocardiography (ECG), pulse oximetry, photoplethysmography (PPG), and bioimpedancemetry. As a result, the device achieves the claimed technical effect of delivering detailed diagnostic insights, such as ECG waveform analysis, real-time monitoring of blood oxygen saturation levels, and assessment of vascular and tissue characteristics. This integrated functionality facilitates early detection of potential complications or critical conditions affecting the cardiovascular system, thereby enhancing preventive care and real-time physiological assessment.

While the disclosed technology has been described in detail with reference to specific exemplary embodiments, including particular apparatuses and methods, it will be understood by those skilled in the art that various modifications, substitutions, changes, and equivalents may be made without departing from the scope of the invention. The embodiments described herein are illustrative and not intended to be limiting. The scope of the invention, including apparatus and method aspects, is defined by the appended claims and is intended to cover all such modifications and equivalents that fall within the true spirit and scope of the invention as defined by those claims.

What is claimed is:

1. An integrated electrocardiographic mobile complex comprising:
a housing;
at least one electrode disposed on the housing;
a microcontroller disposed within the housing, the microcontroller comprising:
an analog-to-digital converter configured to convert analog signals from the at least one electrode into digital signals;
a cardiomodule functionally connected to the at least one electrode and configured to receive electrical signals of a heart;
a pulse oximeter module configured to receive optical signals for determining blood oxygen saturation (SpO₂) levels;
an impedance measurement module;
a digital accelerometer;
a temperature measurement module comprising a temperature sensor;
a light indicator module; and
a tactile indication module;
a memory unit configured to store data from the microcontroller;
a power supply unit; and
a wireless transmit/receive unit configured to transfer data to and from an external device, the wireless transmit/receive unit comprising:
a Bluetooth module; and
an RF module configured to transmit and receive signals at a distance of at least one kilometer, and to generate up to one hundred time-and-frequency communication channels;
wherein the microcontroller is configured to:
perform diagnostics and obtain real-time synchronized complex electrocardiographic data, pulse oximetry data, photoplethysmography data, and bioimpedancemetry data;
convert analog signals from the cardiomodule into digital data via the analog-to-digital converter;
receive digital signals from the pulse oximeter module, impedance measurement module, and the temperature sensor; and
compile and transfer the data as a data package to the wireless transmit/receive unit;
wherein the pulse oximeter module is configured to:
process low-noise analog signals; and
enter a software-controlled standby mode while maintaining continuous connection to the power supply unit;
and wherein one of the electrodes on the front surface of the housing is functionally connected to the cardiomodule for receiving cardiac signals, and another of the electrodes comprises a sensor connected to the pulse oximeter module for parallel recording of optical signals.

2. The integrated electrocardiographic mobile complex of claim 1, wherein the memory unit comprises a non-volatile memory with an SPI interface configured to store data in the absence of communication between the portable hardware-software ECG complex and an external device, the external device comprising a receiving docking station and/or a mobile phone.

3. The integrated electrocardiographic mobile complex of claim 1, wherein the cardiomodule comprises an analog interface configured to measure the patient's biopotentials and to amplify and filter the signal under conditions of high noise, including artifacts caused by patient muscle movement and external electromagnetic interference, from a single cardiogram lead.

4. The integrated electrocardiographic mobile complex of claim 1, wherein the impedance measurement module is configured to measure the complex impedance values of the patient's body and to transmit the measured data to the microcontroller via an SPI bus.

5. The integrated electrocardiographic mobile complex of claim 1, wherein the temperature measurement module comprises a digital integrated circuit including a digital pyrometer sensor configured to measure contact or non-contact temperature of the patient's fingers.

6. The integrated electrocardiographic mobile complex of claim 1, wherein the pulse oximeter module comprises a digital integrated circuit including an optical interface configured to display information related to the pulse wave and the blood oxygen level.

7. The integrated electrocardiographic mobile complex of claim 1, wherein the tactile indication module comprises an oscillation generator coupled to the housing and configured to generate tactile signals for the user during periods of active physical activity or when visual or other device status indicators are unavailable.

8. The integrated electrocardiographic mobile complex of claim 1, wherein the light indicator module comprises RGB LEDs configured to display information regarding the status and operating modes of the portable hardware-software ECG complex.

9. The integrated electrocardiographic mobile complex of claim 1, wherein the wireless transmit/receive unit is further configured to dynamically switch between the Bluetooth module and the RF module based on signal strength, battery level, or data priority.

10. The integrated electrocardiographic mobile complex of claim 1, wherein the digital accelerometer is configured to detect and classify user body posture and physical activity levels, and to transmit corresponding motion metadata to the microcontroller for inclusion in the data package.

11. The integrated electrocardiographic mobile complex of claim 1, wherein the housing comprises a waterproof or splash-resistant enclosure conforming to at least IP65 standards, and is shaped ergonomically for prolonged skin contact.

12. The integrated electrocardiographic mobile complex of claim 1, wherein the microcontroller is further configured to initiate a self-test diagnostic sequence upon startup or user request to verify the operational status of each functional module.

13. The integrated electrocardiographic mobile complex of claim 1, wherein the memory unit is further configured to buffer real-time data for a predetermined duration in the event of temporary wireless communication loss.

14. The integrated electrocardiographic mobile complex of claim 1, wherein the power supply unit comprises a rechargeable lithium-polymer battery and an energy management circuit configured to prioritize power delivery based on module usage frequency and criticality.

15. The integrated electrocardiographic mobile complex of claim 1, wherein the light indicator module is configured to provide visual alerts in response to abnormal physiological measurements, including heart rate anomalies, low SpO2 levels, or elevated body temperature.

16. A method of performing physiological diagnostics using an integrated electrocardiographic mobile complex, the method comprising:
providing a mobile complex comprising a housing, electrodes, a microcontroller, a memory unit, a wireless transmit/receive unit, and a power supply unit;
receiving analog electrical signals from at least one electrode placed in contact with a subject's skin;
processing the analog electrical signals in a cardiomodule to obtain cardiac data;
receiving optical signals from a sensor in communication with a pulse oximeter module to determine a blood oxygen saturation (SpO₂) level;
receiving signals from an impedance measurement module to determine bioimpedance;
receiving signals from a digital accelerometer and a temperature sensor;
converting the analog signals into digital signals via an analog-to-digital converter;
aggregating digital signals from the cardiomodule, pulse oximeter module, impedance measurement module, temperature sensor, and accelerometer;
generating a data package including the aggregated physiological data; and
transmitting the data package to an external device via a wireless transmit/receive unit comprising a Bluetooth module and an RF module.

17. The method of claim 16, further comprising:
operating the RF module to transmit data over a distance of at least one kilometer.

18. The method of claim 16, further comprising:
placing the pulse oximeter module into a standby mode via software while maintaining electrical connection to the power supply unit.

19. The method of claim 16, further comprising:
synchronizing in real time the data received from the cardiomodule, pulse oximeter module, impedance measurement module, accelerometer, and temperature sensor.

20. The method of claim 16, wherein:
the generated data package includes time-stamped physiological parameters corresponding to electrocardiography, photoplethysmography, pulse oximetry, and bioimpedancemetry.
